(19) United States
(12) Patent Application Publication
(10) Pub. No.: US 2009/0181291 A1
(43) Pub. Date: Jul. 16, 2009

(54) Surgical Instrument With Lithium Ion Energy Source Including Phosphates

(76) Inventors: Lucian R. Lewis, II, Charlottesville, VA (US); Daniel Geisert, Charlottesville, VA (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 12/351,341

(22) Filed: Jan. 9, 2009

Related U.S. Application Data
(60) Provisional application No. 61/020,555, filed on Jan. 11, 2008.

(51) Int. Cl. H01M 2/10; A61L 2/07

(52) U.S. Cl. 429/100; 422/26

(57) ABSTRACT

Lithium ion batteries which contain phosphates can serve as sterilizable power sources for use in surgical tools. Battery packs containing lithium ion batteries that contain phosphates are able to survive lengthy autoclaving cycles, where lithium ion batteries without phosphates cannot survive such cycles. Thus, a surgical tool can be provided sterilizable power source when phosphate containing lithium ion batteries are used.

### CROSS-REFERENCE TO RELATED APPLICATIONS

The present application claims priority to U.S. Provisional Application 61/020,555 filed Jan. 11, 2008, and the complete contents of this application is herein incorporated by reference.

### BACK GROUND OF THE INVENTION

1. Field of the Invention

The invention is generally related to surgical instruments and, more particularly, to battery sources which used in surgical instruments which must withstand a flash autoclave cycle between uses while avoiding catastrophic failure.

2. Background Description

Battery powered surgical instruments, including saws, drills, etc., have been in use for a number of years. Historically, the battery cells used in these instruments have been nickel based chemistries such as Nickel-Cadmium (Ni-Cad) or Nickel-Metal Hydride (NiMH). The reason for the Nickel based cells predominating in surgical instrument applications are that they provide high current output for driving motors in surgical instruments, and they are able to be autoclave sterilized with a four minute “Flash” autoclave cycle if enclosed in a properly designed plastic housing. The nickel chemistries are stable enough to fail safely if they are mistakenly autoclaved for a long cycle (e.g., 35 minutes).

Most consumer electronic devices have migrated away from nickel based chemistries to lithium ion cells because of lithium ion's much higher energy density (Watt-hrs/kg), which allows for long run times from a light weight battery. While the weight advantage of lithium ion batteries would be attractive for use in surgical instruments, the tendency for conventional (e.g., cobalt, manganese, or nickel-cobalt-manganese cathodes) lithium ion cells to fail violently when overheated, overloaded or are damaged has prevented their use in surgical instrument applications.

### SUMMARY OF THE INVENTION

It is an exemplary object of this invention to provide a sterile method for providing power to a surgical tool, where the tool employs a lithium ion phosphate battery.

It is another exemplary object of this invention to provide surgical tools and battery packs for surgical tools that employ a lithium ion phosphate battery where the battery is safe from catastrophic failure resulting from an autoclave treatment.

According to the invention, lithium ion batteries that include phosphate have been shown to not be prone to catastrophic failure upon sterilizing battery packs containing such batteries in an autoclave.

### BRIEF DESCRIPTION OF THE DRAWINGS

The foregoing and other objects, aspects and advantages will be better understood from the following detailed description of the preferred embodiments of the invention with reference to the drawings, in which:

FIGS. 1a-1c show exemplary battery powered surgical handpieces;

FIG. 2 shows an exemplary battery charging station;

FIG. 3 shows an exemplary lithium ion phosphate based battery which can be used in the practice of the invention; and

FIG. 4 shows an exemplary aseptic battery pack.

### DETAILED DESCRIPTION

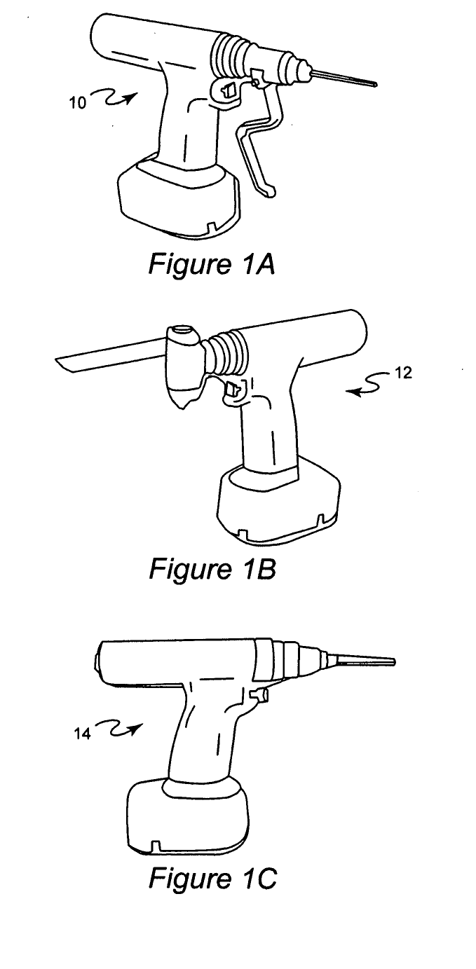

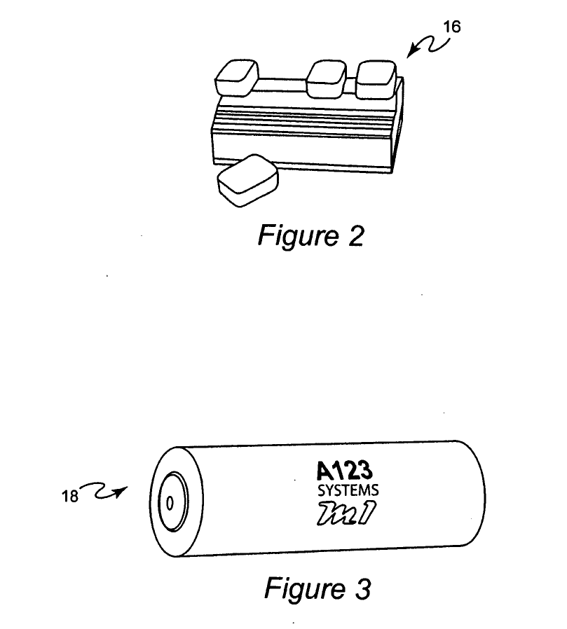

FIGS. 1A-1C respectively show surgical tools in the form of a drill/reamer 10 (FIG. 1A), oscillating saw 12 (FIG. 1B), and reciprocating saw 14 (FIG. 1c). The base of these surgical tools includes a detachable battery pack in which a phosphate containing lithium battery is housed; however, in some embodiments a phosphate containing lithium battery can be housed in the handle or other appropriate portion of the surgical tool. When a battery pack is used, it is selectively detachable from the tool and can be charged in a charging station 16 as shown in FIG. 2, where the contacts used for powering the tool are used for connecting the battery to the charging station 16. FIGS. 1A-1C show examples of the types of surgical tools which can benefit from the invention; however, it should be understood that a wide variety of surgical tools which employ batteries as a power source may also benefit from the invention. In addition, the battery packs shown in FIGS. 1A-1C can have a housing which is separable to allow removal of the lithium ion energy cell, or can have a housing which is fused together after insertion of the lithium ion energy cell.

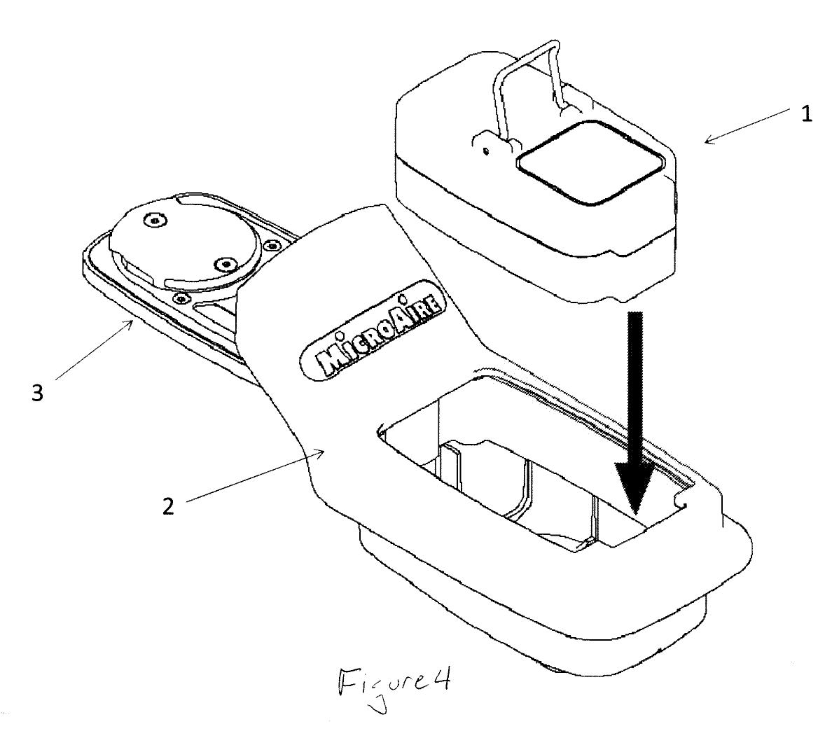

FIG. 3 shows an example of a phosphate containing lithium ion cell 18 which can be used in the practice of the invention. In these cells, the cathode contains phosphate. The form of the lithium ion cell used in the practice of this invention can vary depending on the surgical tool application. A particularly useful material for batteries used in the practice of this invention is a nanoscale alkaline transition metal phosphate having the formula LiMPO₄ where M is one or more transition metals (e.g., LiFePO₄). These nanoscale ion storage materials, when contained in batteries (energy storage cells) provide high energy, and are described in U.S. Patent Publication 2007/0190418 and U.S. Patent Publication 2007/0031732, both of which are herein incorporated by reference.

Surgical tools are used in a sterile environment, and must themselves be sterilized between uses. One mechanism for sterilizing surgical tools is to put them in an autoclave under temperature and pressure conditions and for a period of time which kills bacterial organisms and renders the surfaces of the tools sterile for safe use in surgery. Typical dwell times for autoclavable battery packs is 3-4 minutes in an autoclave under temperature and pressure conditions suitable for sterilization (e.g., exposure to at least 270 degrees Fahrenheit for at least 3-4 minutes under pressure). Lithium ion batteries used in non-surgical applications typically do not require sterilization, and lithium ion batteries that do not include phosphate are subject to catastrophic failure on autoclaving, making them unsuitable for use in surgical tools.

Lithium ion batteries containing phosphate were obtained from A123 Systems, Inc. (in particular, ANR26650M1 high power lithium cells). Conventional lithium ion batteries which lack phosphate were obtained from Emoli corporation (in particular, IMR 18650 E cells). Fully charged cells were put in a Ritter M7 Speedclave at 270 degrees Fahrenheit for 30 minutes to determine if the cells could withstand autoclave exposure. At the end of the cycle, the pressure of the autoclave was released, the cells were cooled, and the cells were examined. The top and bottom of the non-phosphate containing lithium cells were blown off and material form inside the cells leaked out considerably after a single autoclave cycle. The non-phosphate containing lithium cells were not charged and not suitable for use in a surgical tool after an autoclave procedure (i.e., they cannot withstand the steam, heat, and pressure conditions of an autoclave). In contrast, lithium ion cells containing phosphate which were housed in a battery pack did not explode, they maintained their charge (e.g. autoclaved cells had voltages of 3.37-3.38 V; and they maintained an available capacity near tolerance (2.275 Ah on a 2.3 Ah rating) after a 30 minute cycle. Phosphate containing lithium ion cells which were cycled for one hour in an autoclave did not explode, but did have minor amounts of chemical leakage. Based on these results, phosphate containing lithium ion batteries used in surgical tools or battery packs for surgical tools can withstand autoclave temperatures, pressures and steam conditions which would enable them to be used in the surgical environment, which requires sterilization between uses.

FIG. 4 shows an exemplary aseptic battery pack 1 containing lithium ion nanophosphate batteries which includes an autoclavable transfer shield 2 and autoclavable battery housing 3. Aseptic battery packs are used on a variety of surgical tools and typically use nickel metal hydride power sources. In the practice of this invention, lithium ion nanophosphate batteries will be used as the power source. Aseptic battery packs are generally not autoclaved as part of normal usage; however, there is a substantial risk of them being autoclaved accidentally. The tests noted above demonstrate that lithium ion batteries containing phosphate will not explode in autoclave and do not pose a fire hazard if autoclaved accidentally.

While the invention has been described in terms of its preferred embodiments, those skilled in the art will recognize that the invention can be practiced with modification within the spirit and scope of the appended claims.

1. A sterile method for providing power to a surgical tool; comprising the steps of:
autoclaving a surgical tool or battery pack for a surgical tool for a time and at a temperature and pressure sufficient to sterilize the surgical tool or battery pack, wherein the surgical tool or battery pack includes a lithium ion battery that includes phosphate; and
powering the surgical tool the lithium battery after said step of autoclaving.

2. The sterile method of claim 1 wherein said lithium ion battery includes LiMPO₄, where M is one or more transition metals.

3. The sterile method of claim 2 wherein M is Fe.

4. The sterile method of claim 1 wherein said step of autoclaving is performed at a temperature of at least 270 degrees Fahrenheit and for a period of at least 3-4 minutes.

5. A sterilizable battery powered surgical tool, the improvement comprising a lithium ion battery wherein a cathode of the battery includes phosphate and where the lithium ion battery.

6. A sterilizable battery pack for a surgical tool, comprising:
a housing; and
a lithium ion battery with a cathode which includes phosphate, said lithium ion battery being positioned within said housing.

7. An aseptic battery pack safe against accidental autoclaving procedures, comprising:
a housing; and
a lithium ion battery with a cathode which includes phosphate, said lithium ion battery being positioned within said housing.
